(19) United States
(12) Patent Application Publication
(10) Pub. No.: US 2026/0108396 A1
(43) Pub. Date: Apr. 23, 2026

(54) DISPOSABLE AND REUSABLE DIAPER

(71) Applicant: Hetvi Lapasia, Mumbai (IN)

(72) Inventor: Hetvi Lapasia, Mumbai (IN)

(21) Appl. No.: 18/923,390

(22) Filed: Oct. 22, 2024

(51) Int. Cl. A61F 13/49; A61F 13/505; A61F 13/56

(52) U.S. Cl. CPC A61F 13/49006; A61F 13/505; A61F 13/565

(57) ABSTRACT

A disposable and reusable diaper 100 comprising a reusable pant 200, constructed from soft, stretchy cotton material further comprising front panel elevation, back panel elevation and fastening components and a disposable natural insert 300 disposed within and releasably attached to the reusable pant 200, made configured to absorb and retain body exudates of an infant, further comprising a front waist area 324, a back waist area 320, and a crotch area 322 interconnecting the front and back waist areas, an outer layer 330, an inner layer 334, an absorbent core 332, a fabric pouch 336 and a fastening components 346. The fabric pouch which serves as a convenient storage compartment for a roll of one or two wet wipes comprises a sealing flap 338, a hollow tube 340, wet wipes 342 and a thin thread 344.

### FIELD OF THE INVENTION

The present invention relates to a diapering solution for newborns, infants and toddlers. More particularly, it relates to a disposable and reusable diaper.

### BACKGROUND OF THE INVENTION

Diapering solutions have been an integral part of infant care, with the market historically dominated by two main types: disposable diapers and reusable cloth diapers. Disposable diapers are widely favoured by parents for their convenience. They are designed for single use and are highly absorbent, ensuring that babies remain dry for extended periods. These diapers are typically made from synthetic materials, including a waterproof outer layer and an absorbent core, making them reliable for leakproof performance. The convenience of disposing of the diaper after each use, without the need for washing, has made disposable diapers the go-to choice for the majority of parents. Reusable cloth diapers, on the other hand, are seen as an eco-friendlier option. Made from natural fibers such as cotton, hemp, or bamboo, these diapers can be washed and reused multiple times, reducing waste.

However, reusable cloth diapers are often perceived as inconvenient by busy parents. The need to regularly wash, dry, and prepare these diapers adds to the already demanding responsibilities of parenthood. As a result, many parents opt for disposable diapers, prioritising convenience over sustainability in their daily routine.

Despite the widespread adoption of these traditional diapering solutions, they present several challenges that have led to growing dissatisfaction among parents:

Environmental Impact of Disposable Diapers: The widespread use of disposable diapers has led to significant environmental concerns. 95% of diapers used are disposable, contributing to the approximately 20 billion diapers that are thrown away globally each year. These diapers take hundreds of years to decompose, filling landfills and contributing to pollution. Parents are increasingly aware of this impact and are seeking more sustainable alternatives.

Parental Stress and Anxiety: Choosing the right diaper is a significant source of stress and anxiety for 67% of parents. They feel overwhelmed by the need to trial and test multiple diaper brands to find the one that suits their baby perfectly. This process is not only time-consuming but also emotionally draining, adding to the overall burden of parenting.

Comfort Issues: Traditional disposable diapers, made of polypropylene plastic often include elastic components to ensure a snug fit. However, these elastics can cause discomfort, leading to red marks and irritation on the baby's sensitive skin. Many parents report that because of the elastic and the plastic material in the diaper, their babies develop red marks around the joints, causing concern and discomfort for both the baby and the parents.

Frequency of Diaper Changes: Parents typically change diapers 4 to 6 times per day, which amounts to approximately 6,000 diapers per baby over the diapering period. The sheer number of diapers used, combined with the inconvenience of frequent changes, particularly with non-disposable options, adds to the overall stress and burden on parents.

Given these challenges, there is a clear need to rethink the diapering experience—to create a solution that addresses the critical concerns of comfort, sustainability, and functionality for both parents and babies. The present invention aims to fulfil this need by introducing a disposable and reusable diaper for newborns, infants and toddlers.

### PRIOR ART

U.S. Pat. No. 7,875,014B2: A disposable absorbent garment for wear about a wearer's waist includes a garment shell and an absorbent assembly constructed to take in and retain body exudates released by the wearer, the absorbent assembly including an inner waistband. The absorbent assembly may be permanently detached from the garment shell. The garment shell has limited attachment to the absorbent assembly at front and rear attachment zones. The inner waist belt provides tension on each side of the absorbent assembly to provide effective coverage of the wearer.

U.S. Pat. No. 9,095,478B2: A wearable absorbent article comprising a reusable outer cover and a disposable absorbent insert is provided. The disposable absorbent insert comprises a substantially liquid impermeable backsheet. The reusable outer cover comprises a bending stiffness peak force, measured according to the Bending Stiffness Test. The bending stiffness peak force of the reusable outer cover is less than or equal to 0.20 Newtons.

### OBJECTS OF THE INVENTION

The primary object of the invention is to provide a disposable and reusable diapering solution for newborn, infants and toddlers.

Another object of the present invention is to reduce environmental impact associated with diapering by minimising waste and promoting eco-friendly practices.

Yet another object of the present invention is to enhance the comfort of the baby by reducing the incidence of red marks and irritation on the baby's sensitive skin and make it comfortable.

Yet another object of the present invention is to provide a diapering solution that is convenient for busy parents and alleviates parental stress and anxiety related to choosing the right diaper.

Yet another object of the present invention is to provide a fashionable and comforting diapering option that allows the pants to serve as a stylish and functional garment, eliminating the need for the baby to wear multiple layers of clothing.

### SUMMARY OF THE INVENTION

This invention offers an advanced diapering solution that integrates the convenience of disposable inserts with the sustainability of reusable cloth pants, providing a practical and eco-friendly alternative for infant and toddler care. The reusable pant is crafted from soft, stretchy cotton, designed for breathability and comfort. It features adjustable waistbands and snug-fitting leg openings, achieved through press buttons and fasteners, ensuring a customizable fit that minimises leaks. The pants structure, with front and back panel elevations and crotch flaps, provides full coverage while allowing for easy adjustment to suit the baby's body shape.

The core innovation lies in the disposable natural insert, which fits inside the reusable pant independently and is kept on the surface of the pant. This insert is made up of three layers: a waterproof outer layer that prevents leakage, an absorbent core made from natural fibers like hemp or bamboo for maximum fluid absorption, and a soft inner layer that wicks moisture away from the baby's skin to keep it dry and irritation-free. The insert fastens securely to the reusable pant, ensuring it stays in place during movement, further enhancing its leak-proof capabilities.

A unique feature of the design is the integrated fabric pouch located at the back of the disposable insert. This pouch is designed to store a roll of one or two wet wipes for a single diaper, making diaper changing more convenient. The wipes are pre-perforated for easy dispensing, and the pouch's sealing flap ensures the remaining wipes stay moist and fresh. Both are disposed after use and they are made of natural biodegradable disposable material. This addition streamlines the diapering process by keeping all essentials within reach.

The solution addresses several key problems commonly associated with traditional diapers. The adjustable fit and waterproof outer layer of the insert prevent leaks, while the use of natural, hypoallergenic materials promotes skin health by reducing moisture and the risk of diaper rash. Additionally, the diaper system's sustainability is a major advantage, as the reusable pant reduces waste, and the disposable insert is made from biodegradable materials, providing an eco-friendly alternative to conventional disposable diapers.

By combining the reusability of cloth diapers with the convenience of disposable inserts, the invention offers parents a practical, eco-conscious solution. The design also prioritises comfort and convenience, with features such as adjustable fasteners, a leak-proof absorbent core, and a built-in wet wipe pouch that simplify diaper changes while ensuring the baby's skin stays dry and protected. This innovation stands out as an effective, environmentally responsible approach to modern diapering needs.

### BRIEF DESCRIPTION OF THE DRAWINGS

FIG. 1 of the present invention illustrates an open plan of the disposable and reusable pant.

FIGS. 2a and 2b illustrate the front and back panel elevation of the present invention.

FIG. 3 illustrates the disposable natural insert of the present invention.

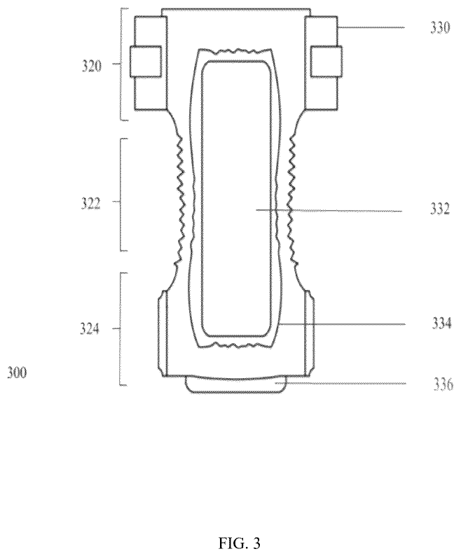

FIG. 4 illustrates the placement of the fabric pouch on the present invention.

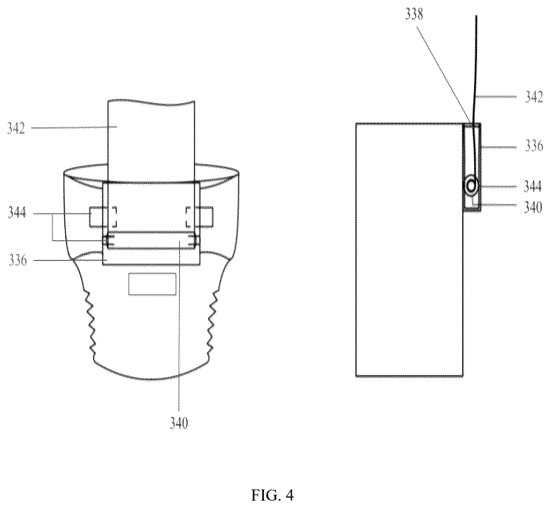

### DETAILED DESCRIPTION OF THE INVENTION

Before the present invention is described, it is to be understood that this invention is not limited to methodologies described, as these may vary as per the person skilled in the art. It is also to be understood that the terminology used in the description is for the purpose of describing the particular embodiments only and is not intended to limit the scope of the present invention. Throughout this specification, the word “comprises”, or variations such as “comprises” or “comprising”, will be understood to imply the inclusion of a stated element, integer or step, or group of elements, integers or steps, but not the exclusion of any other element, integer or step, or group of elements, integers or steps. The use of the expression “at least” or “at least one” suggests the use of one or more elements or ingredients or quantities, as the use may be in the embodiment of the invention to achieve one or more of the desired objects or results. Various embodiments of the present invention are described below. It is, however, noted that the present invention is not limited to these embodiments, but rather the intention is that modifications those are apparent are also included.

The present invention relates to the field of infant and toddler care, specifically to diapering solutions. More particularly, it pertains to a disposable and reusable diaper that combines the convenience of natural disposable inserts with the sustainability of reusable cloth pants. The invention aims to address the challenges associated with traditional diapering options by providing a more comfortable, eco-friendly, and functional solution for both parents and babies.

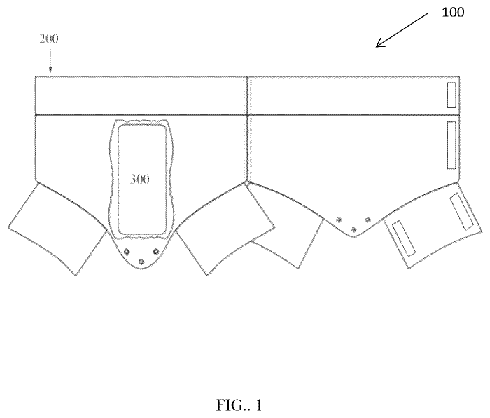

FIG. 1 illustrates an open plan of the present invention, a disposable and reusable diaper 100 comprising a reusable pant 200 constructed from soft, stretchy cotton and a disposable natural insert 300 disposed within and releasably attached to reusable pant 200, constructed to take in and retain body exudates by the infants providing leakproof performance.

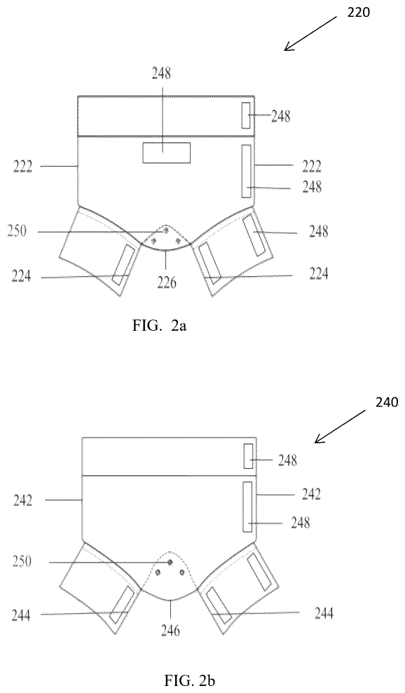

According to another embodiment. FIGS. 2a and 2b illustrates the reusable pant 200 comprising a front panel elevation 220, having side margins 222, inner leg margins 224 and crotch flap 226 forms the front portion of the reusable pant 200 and a back panel elevation 240, having side margins 242, inner leg margins 244 and crotch flap 246 forms the back portion of the reusable pant 200.

According to another embodiment, the side margins 222, 242 of the front and back panel elevations 220, 240 of the reusable pant 200 are overlapped and attached to each other with the help of Velcro which defines the side seam, as shown in FIG. 2a, and the waist band opening is on the lines of the side seam which is where the Velcro is during wear. The inner leg margins 224, 244 are overlapped and attached to each other to broadly define the leg openings, ensuring snug fit around the infant's thigh minimising the risk of leaks. The crotch flap 226, 246 of the front and back panel elevations 220, 240 are used to adjust the size and fitting around the crotch area 322 according to the baby's comfort, through the press button configuration 250.

According to another embodiment, the front and back panel elevations 220, 240 of the reusable pant 200 can be releasably attached to each other at the respective side margins 222, 242 and inner leg margins 224, 244 of the panel elevation, through a fastening component 228 attached to the side margin 222 and inner leg margin 224 of the front panel elevation 220 and is adapted for refastenable engagement with a complementary fastening component 248 attached to the side margin 242 and inner leg margin 244 of the back panel elevation 240. The fastening components 228, 248 can comprise any refastenable fasteners suitable for garments, such as Velcro or the like. The other laterally opposite side margins 222, 242 of the front and back panel elevations 220, 240 are permanently secured through stitching, thereby facilitating the reusable pant 200 to fully open and allow for easy dressing and removal on the baby. The front and back panel elevation 220, 240 is equipped with press button configuration 250 around the waistband, allowing parents to adjust the size of the waist opening to ensure a snug and comfortable fit for the baby.

According to another embodiment, the disposable natural insert 300 comprises a front waist area 324, a back waist area 320, a crotch area 322 interconnecting the front and back waist areas. The front waist area 324, when the diaper is worn by the infant, is positioned in front of the infant while the back waist area 320 is positioned on the back of the infant and the crotch area 322 is positioned in between the legs of the infant.

According to another embodiment, the disposable natural insert 300 is suitably rectangular in shape, but it is understood that the disposable natural insert 300 may be other than rectangular, such as hourglass-shaped, T-shaped, I-shaped or other suitable shape without departing form the scope of the invention.

According to another embodiment, the disposable natural insert 300 comprises an outer layer 330, an absorbent core 332, an inner layer 334 and a waterproof disposable fabric pouch 336. The inner layer 334 is in superposed relationship with the outer layer 330 and the absorbent core 332 is disposed between the outer layer 330 and the inner layer 334. The outer layer 330 of the disposable natural insert 300 is made of a waterproof material or liquid impermeable material, such as but not limited to biodegradable polymer or laminated natural fabric, designed to prevent leakage. The outer layer 330 serves as a protective barrier, ensuring that moisture is contained within the insert and does not seep through to the outer garments or surroundings.

According to another embodiment, the absorbent core 332 is crafted from natural fibers such as hemp, bamboo pulp or blend of these materials, which have excellent absorbency, antibacterial properties, and biodegradability. The core 332 is engineered with a high-density fiber structure that can efficiently absorb and retain fluids, keeping the baby's skin dry and reducing the risk of diaper rash. The absorbent core 332 is positioned between the outer layer (330) and the inner layer 334, which can be joined together by any suitable means such as natural adhesives, ultrasonic bonds, thermal bonds, or the like.

According to another embodiment, the inner layer 334 is made from a soft, breathable, and hypoallergenic material such as organic cotton or a bamboo fiber or a blend. The natural fibers of organic cotton are also less likely to cause irritation or allergic reactions, making it ideal for use in diapers. Bamboo fiber is highly absorbent and can wick moisture away from the skin more effectively than many other natural materials, it also possesses thermo-regulating properties, keeping the baby cool in warm weather and warm in cooler conditions. The inner layer 334 is liquid permeable, overlying on the outer layer 330 and absorbent core 332 allows liquid to pass through to the absorbent core while keeping the infant's skin dry.

According to another embodiment, the fabric pouch 336 is in superposed relationship with outer layer 336, placed at the front waist area 324 on the outer layer 330 of the disposable natural insert 300. The pouch 336 is at the back of the natural insert but comes on the front side of the baby when worn. The fabric pouch is designed to be flexible, thin and durable allowing for a non-intrusive feel while worn. The material of the fabric pouch 336 is also biodegradable, ensuring that the overall product remains eco-friendly. The fabric pouch 336 is suitably rectangular in shape, but it is understood that the fabric pouch may be other than rectangular shape, such as hourglass, I-shaped or other suitable shape, tailored to fit comfortably on the front waist area 324 of the disposable natural insert 300. The fabric pouch 336 is stitched by a thin thread 344 that secures it to the outer layer 330 of the disposable natural insert 300, ensuring it remains in place without shifting during movement. The dimensions of the fabric pouch 336 are sufficient to hold the rolled a roll of one or two wet wipes but slim enough to avoid affecting the overall diaper fit.

According to another embodiment, the fabric pouch 336 comprises a sealing flap 338, a hollow tube 340 and a continuous separable wet wipe 342.

According to another embodiment, the sealing flap 338 positioned on the top of the fabric pouch 336 enables an opening which ensures easy retrieval of wet wipes by the user while keeping the wipes fresh and moist inside the fabric pouch 336.

The roll of one or two wet wipes 342 are arranged in a roll around the hollow tube 340 which serves as a spindle for smooth dispensing. The wet wipes are perforated sections to allow easy separation from the roll by the user. When the user pulls a wet wipe from the fabric pouch 336, the perforation ensures that only one wipe is dispensed at a time, with minimal effort. The user can access the wipes through the sealing flap 338 of the fabric pouch 336 and after each use the opening can be sealed again using the flap to prevent drying of the remaining wipes.

According to another embodiment, the hollow tube 340 has a central cavity through which air can circulate, reducing the chance of condensation or excess moisture building up inside the fabric pouch 336 ensuring that the wipes 342 remain moist but not overly saturated. The hollow tube 340 is secured in place by the thin thread 344 that ties the outer edges of the hollow tube to the fabric pouch 336 enabling the roll of one or two wet wipes to rotate freely around the hollow tube.

According to another embodiment, the thin thread 344 helps in securing the fabric pouch 336 to the outer layer 330 by way of stitching and the hollow tube 340 to the fabric pouch 336. The thin threads 344 hold the hollow tube 340 securely while allowing the roll of wipes to unwind smoothly as needed from the fabric pouch 336. Also, the thin thread 344 holds the fabric pouch securely while allowing accommodation of the hollow tube 340 around which the wet wipes are wound. The thin thread 344 is made of natural, biodegradable fibers to maintain the eco-friendliness of the overall product.

According to another embodiment, the disposable natural insert 300 is placed inside the reusable pant 200, with its front waist area 324 positioned towards the front panel elevation 220 of the pant and its back waist area 320 towards the back panel elevation 240 of the pant. The crotch area 322 of the insert fits snugly between the infant's legs, aligning with the crotch flaps 226, 246 of the pant. The fastening components of the disposable natural insert positioned on the outer layer 330 of the disposable natural insert 300 at front waist area 324 and back waist area 320 engage with the complementary fastening components of the reusable pant 200 positioned on the inside of the front panel elevation 220 and back panel elevation 240 respectively, secures the disposable natural insert 300 in place, ensuring it remains aligned and stable during wear, even with the baby's movements. The snug fit created by the fastening component of the insert within the reusable pant ensures that there are no gaps between the insert and the pant, minimising the risk of leaks.

### ADVANTAGES

Eco-friendly and Sustainable: The combination of reusable and disposable components significantly reduces the environmental impact by minimising waste compared to traditional disposable diapers. The reusable pant can be washed and reused, promoting eco-friendly diapering practices. The pant can be worn throughout the day and that reduces the number of diapers used. The natural insert can be changed when needed.

Comfort and Skin Protection: The use of soft, natural materials in the disposable insert, combined with the reusable pants design, tends to reduce the incidence of red marks and irritation on the baby's sensitive skin, enhancing overall comfort for newborns, infants, and toddlers.

Convenience for Parents: The hybrid diapering solution offers a convenient option for busy parents by simplifying the diapering process. The ease of use of the disposable insert, paired with the reusable pant, provides peace of mind, reducing parental stress and anxiety when choosing the right diaper and being eco-conscious which most parents want to.

Cost-effective: By incorporating a reusable pant with disposable inserts, parents can save money over time compared to the continuous purchase of fully disposable diapers. The reusable element provides a long-term solution while the disposable inserts are easy to replace when needed.

Leak-Proof Design: The advanced fastening system and careful material selection provide reliable leak protection. The snug fit between the insert and the pant ensures that the diaper stays securely in place, reducing the risk of leaks even with active babies.

Stylish and Functional: The reusable pant is designed to be both functional and fashionable, allowing it to serve as a stylish garment. This eliminates the need for additional clothing layers, making it a versatile wardrobe option for the baby.

Sustainable Diapering Solution: The diapering system promotes the use of natural, biodegradable materials in the disposable insert, further reducing the environmental footprint and supporting sustainable practices in baby care.

The primary components of the present invention are set forth in the Table below:

TABLE

| Number | Name of the component |
|---|---|
| 100 | disposable and reusable diaper |
| 200 | reusable pant |
| 220 | front panel elevation |
| 222 | side margins of front panel elevation |
| 224 | inner leg margins of front panel elevation |
| 226 | crotch flap of front panel elevation |
| 240 | back panel elevation |
| 242 | side margins of back panel elevation |
| 244 | inner leg margins of back panel elevation |
| 246 | crotch flap of back panel elevation |
| 248 | fastening component of the reusable pants |
| 250 | press button configuration |
| 300 | disposable natural insert |
| 320 | back waist area of the insert |
| 322 | crotch area of the insert |
| 324 | front waist area of the insert |
| 330 | outer layer of the insert |
| 332 | absorbent core |
| 334 | inner layer of the insert |
| 336 | waterproof disposable fabric pouch |
| 338 | sealing flap |
| 340 | hollow tube |
| 342 | a roll of one or two separable wet wipes |
| 344 | thin thread |
| 346 | fastening component |

What is claimed is:

1. A disposable and reusable diaper, comprising:
a reusable pant constructed from soft, stretchy cotton material, the reusable pant comprising of a front panel elevation having laterally opposite side margins, inner leg margins, and a crotch flap forming the front portion of the reusable pant;
a back panel elevation having laterally opposite side margins, inner leg margins, and a crotch flap forming the back portion of the reusable pant;
a fastening component placed at side margins, inner leg margins and top of the front panel elevation and back panel elevation;
a disposable natural insert, disposed within and releasably attached to the reusable pant, the insert configured to absorb and retain body exudates of an infant, the insert comprising of a front waist area, a back waist area, and a crotch area interconnecting the front and back waist areas;
an outer layer made of a liquid impermeable material;
an inner layer made from a breathable and hypoallergenic material providing comfort and reduced risk of skin irritation;
an absorbent core disposed between the outer layer and an inner layer;
a fabric pouch positioned on the outer layer of the front waist area;
a fastening component positioned on the outer layer of the disposable natural insert at the front waist area and the back waist area;
the fabric pouch made of biodegradable material comprises of a sealing flap which is positioned on the top of the fabric pouch to enable an opening which ensures easy retrieval of wet wipes by the user while keeping the wipes fresh and moist inside the fabric pouch;
a hollow tube designed with a central cavity, allowing air to circulate and thereby reducing the likelihood of condensation or excess moisture build-up inside the fabric pouch wherein the hollow tube is secured in place by the thin thread that ties outer edges of the hollow tube to the fabric pouch;
a roll of one or two wet wipes which are perforated, arranged in a roll around the hollow tube which serves as a spindle for smooth dispensing; and
a thin thread made from biodegradable material helps in securing the fabric pouch and the hollow tube to the outer layer by way of stitching.

2. The disposable and reusable diaper as claimed in claim 1, wherein the reusable pant comprises fastening components at the side margins and inner leg margins of the front and back panels, wherein the fastening components are refastenable and adapted to engage with complementary fastening components to form side seams and leg openings.

3. The disposable and reusable diaper as claimed in claim 1, wherein the disposable natural insert comprises fastening components positioned on the outer layer of the disposable natural insert at the front waist area and the back waist area engage with complementary fastening components of the reusable pants securing the disposable natural insert in place within the reusable pant, ensuring alignment and stability of the insert during wear.

4. The disposable and reusable diaper as claimed in claim 1, wherein the absorbent core of the disposable natural insert is made of natural fibers, including but not limited to hemp, bamboo pulp, or a combination thereof, providing absorbency and biodegradability, engineered with a high-density natural fiber structure to enhance fluid retention and reduce the occurrence of diaper rash.

5. The disposable and reusable diaper as claimed in claim 1, wherein the reusable pant further comprises a press button configuration around the waistband for adjustable sizing of the waist opening, and a press button configuration on the crotch flap of the front and back panel elevations to adjust the size and fit around the crotch area for enhanced comfort for the baby.

6. The disposable and reusable diaper as claimed in claim 1, wherein the disposable natural insert is hourglass-shaped or T-shaped to better conform to the infant's body.
